(19) United States
(12) Patent Application Publication
(10) Pub. No.: US 2010/0115638 A1
(43) Pub. Date: May 6, 2010

(54) METHOD FOR INHIBITING THE EXPRESSION OF ENDOGENOUS ERYTHROPOIETIN (EPO)

(76) Inventors: Amina Abina, La Garenne Colombes (FR); Machadiya Muslmani, Le Plessis Robinson (FR); Timothée Cezard, Paris (FR)

Correspondence Address: BUCHANAN, INGERSOLL & ROONEY PC, POST OFFICE BOX 1404, ALEXANDRIA, VA, 22313-1404, US

(21) Appl. No.: 12/520,412

(22) PCT Filed: Dec. 19, 2007
(86) PCT No.: PCT/EP07/64274

§ 371 Date: Dec. 30, 2009

Related U.S. Application Data
(60) Provisional application No. 60/875,530, filed on Dec. 19, 2006.

(51) Int. Cl. A01K 67/033; A61K 38/16; A61K 39/395; A61K 9/127; A61K 45/00; C07K 16/00; A61K 49/00; A61P 7/00

(52) U.S. Cl. 800/8; 514/44 R; 514/12; 424/130.1; 424/450; 424/93.2; 424/93.21; 424/93.7; 530/387.1; 424/9.2

(57) ABSTRACT

The present invention concerns a method for functional inactivation of endogenous erythropoietin (EPO) in a subject comprising the step of (a) co-administrating to said subject at least one agent and at least one erythropoietin protein or derivative, or at least one nucleic acid sequence encoding said erythropoietin protein or derivative, said agent being administrated simultaneously, sequentially or separately with said erythropoietin protein or derivative, or nucleic acid sequence encoding said erythropoietin protein or derivative, wherein said at least one agent and at least one erythropoietin protein or derivative, or at least one nucleic acid sequence encoding said erythropoietin protein or derivative are administrated in an effective amount for triggering the production of neutralizing antibodies against the endogenous erythropoietin (EPO) in said subject; a non human vertebrate which can be obtained by said method and a neutralizing antibody directed against erythropoietin isolated from said subject.

This application claims the priority of the provisional application U.S. 60/875,530 filed on Dec. 19, 2006, which is herein incorporated by reference.

### FIELD OF THE INVENTION

The present invention relates to the field of biology, more precisely to a method for inhibiting the expression of endogenous erythropoietin (EPO) in a subject, and the applications of such a method to therapy and to screening in the animal models having a functional EPO knock-out phenotype.

### BACKGROUND OF THE INVENTION

“Erythropoietin” (EPO) is a glycoprotein hormone which in humans has a molecular weight of about 30-34 kDa. The mature protein has about 166 amino acids, and the oligosaccharide residues comprise about 40% of the weight of the molecule.

For many years, the only clear physiological role of erythropoietin had been its control of the production of red blood cells. Recently, several lines of evidence suggest that erythropoietin, as a member of the cytokine superfamily, performs other important physiologic functions which are mediated through interaction with the erythropoietin receptor (erythropoietin-R). These actions include mitogenesis, modulation of calcium influx into smooth muscle cells and neural cells, production of erythrocytes, hyperactivation of platelets, production of thrombocytes, and effects on intermediary metabolism. It is believed that erythropoietin provides compensatory responses that serve to improve hypoxic cellular microenvironments as well as modulate programmed cell death caused by metabolic stress.

Nevertheless, the absence of any animal model, wherein EPO functions are completely abrogated, complicates the full understanding of EPO biologic functions.

### SUMMARY OF THE INVENTION

The present invention relates to a method for functional inactivation of endogenous erythropoietin (EPO) in a subject comprising the step of (a) co-administrating to said subject at least one agent and at least one erythropoietin protein or derivative, or at least one nucleic acid sequence encoding said erythropoietin protein or derivative, said agent being administrated simultaneously, sequentially or separately with said at least one erythropoietin protein or derivative, or at least one nucleic acid sequence encoding said erythropoietin protein or derivative, wherein said at least one agent and at least one erythropoietin protein or derivative, or at least one nucleic acid sequence encoding said erythropoietin protein or derivative are administrated in an effective amount for triggering the production of neutralizing antibodies against the endogenous erythropoietin (EPO) in said subject.

In a preferred embodiment, the method of the invention comprises the step (a) of administrating to said subject at least one agent selected in the group comprising viruses, parasites, bacteriae and fungunses, which genome comprises at least one nucleic acid sequence encoding for an erythropoietin protein or derivative and regulation sequences necessary to direct the expression of said erythropoietin protein or derivative.

In another preferred embodiment, the method of the invention is a method of screening and identifying compounds acting as EPO agonists, and further comprising the step (b) of administrating at least one compound to the subject.

In still another preferred embodiment, the method of the invention is a method for screening and identifying compounds acting as oxygen transporters, and further comprising the step (b) of administrating at least one compound to said subject.

In still another preferred embodiment, the method of the invention is a method for treating and/or preventing pathology associated with abnormal red blood cells in a subject and further comprises the step of (b) administrating bone marrow cells to said subject enabling the obtaining of normal red blood cells.

The present invention further relates to a non human vertebrate which can be obtained by the method of the invention.

Finally, the present invention also relates to a neutralizing antibody directed against erythropoietin isolated from a subject as described previously.

### BRIEF DESCRIPTION OF THE DRAWINGS

FIG. 1 shows the immunogenicity profile of Rat EPO precursor (SEQ ID NO: 1).

FIG. 2 shows the alignment of rat (SEQ ID NO:1) and mutant (SEQ ID NO:2) sequences. The “*” specify the modification introduce and the “-” a gap introduce.

FIG. 3 shows the immunogenicity profile of Mutant EPO precursor (SEQ ID NO: 2), compared to the endogenous rat sequence. EPO mut precursor ( - - - - ), EPO endogenous rat sequence (—)

FIG. 4 shows the hematocrit evolution in rat blood from rat injected with PBS, AdNull or Ad-EPOMut adenovirus.

FIG. 5 shows the detection of anti-EPO antibodies in sera from Ad-EPOMut treated rats (KO1, KO2 and KO3) and AdNull treated rats (represented in black squares).

FIG. 6 shows the comparison of the titration of anti-EPO antibodies in sera from Ad-EPOMut treated rats (KO1, KO2, and KO3) and AdNull treated rats.

FIG. 7 shows the concentration (ng/ml) of EPO in the serum from Ad-EPOMut, AdNull and PBS treated rats as detected by ELISA at 1 and 2 months following the injection. 1 mo and 2 mo: mean animals analyzed 1 month and 2 months respectively following Adenovirus immunization or control injection.

FIG. 8 shows the immunogenicity profile of EPOmut 1 precursor (SEQ ID NO: 6), compared to the endogenous rat sequence. EPO mut 1 precursor ( - - - - ), EPO endogenous rat sequence (—).

FIG. 9 shows the immunogenicity profile of EPOmut2 precursor (SEQ ID NO: 5), compared to the endogenous rat sequence. EPO mut 2 precursor ( - - - - ), EPO endogenous rat sequence (—).

FIG. 10 shows the hematocrit evolution in rat blood from rat injected with Ad-EPOMut adenovirus.

FIG. 11 shows the hematocrit evolution in rat blood from rat injected with Ad-EPOMut1 adenovirus.

FIG. 12 shows the hematocrit evolution in rat blood from rat injected with Ad-EPOMut2 adenovirus.

FIG. 13 shows the hematocrit evolution in rat blood from rat injected with Ad-EPOrat adenovirus.

FIG. 14 shows the hematocrit evolution (HCT %) in rat blood from rat injected with Ad-EPOmut adenovirus with two round of injection (1^{st}: time 0, and 2^{nd} indicated by arrow).

FIG. 15 shows the hematocrit evolution (HCT %) in rat blood from rat injected with Ad-EPOmut1 adenovirus with two round of injection (1^{st}: time 0, and 2^{nd} indicated by arrow).

### DETAILED DESCRIPTION

The inventors have now discovered that the vaccination of rats and mice with recombinant adenovirus encoding for erythropoietin derivatives induces the production of EPO neutralizing antibodies and enable to obtain an animal model of partial or complete EPO functional inactivation leading to a slight, moderate or profound anemia associated with a partial or complete erythroid differentiation blockade.

This invention provides a new method for inhibiting the expression endogenous erythropoietin (EPO)

Consequently, in one aspect the present invention relates to a method for functional inactivation of endogenous erythropoietin (EPO) in a subject comprising the step of (a) co-administrating to said subject at least one agent and at least one erythropoietin protein or derivative, or at least one nucleic acid sequence encoding said erythropoietin protein or derivative, said agent being administrated simultaneously, sequentially or separately with said at least one erythropoietin protein or derivative, or at least one nucleic acid sequence encoding said erythropoietin protein or derivative, wherein said at least one agent and at least one erythropoietin protein or derivative, or at least one nucleic acid sequence encoding said erythropoietin protein or derivative are administrated in an effective amount for triggering the production of neutralizing antibodies against the endogenous erythropoietin (EPO) in said subject.

As used herein “functional inactivation of endogenous erythropoietin” means the biological inactivation of erythropoietin at the protein level, in opposition with the “conventional knock-out” that is performed at the gene level by homologous recombination. Actually, all the EPO conventional knock-outs have resulted in a lethal phenotype at the embryonic stage. In the method of the invention, the induction of neutralizing antibodies directed against administrated an erythropoietin protein or derivative constitutes the mean to alter the biological activity of the endogenous erythropoietin, which is substantially identical to the administrated erythropoietin or derivative.

Said functional inactivation of endogenous erythropoietin results in the disturbance of erythropoietin functions such as the production of red blood cells, mitogenesis, modulation of calcium influx into smooth muscle cells and neural cells, production of erythrocytes, hyperactivation of platelets, and/or production of thrombocytes.

As an example, the inventors have established that said functional inactivation of erythropoietin results in an anemia, and more specifically in a profound anemia, which is associated with a red cell aplasia with a blood having a “pale” color, wherein the percentage of hematocrit is equal or below 20%, preferably equal or below 10%, as an example equal or below 5%. In the bone marrow, analysis showed a complete abrogation of the erythroid clonogenic progenitors (CFU-E) in the rats with the functional-KO-phenotype. Clinically, the hypoxia induced by the profound anemia was associated with a cutaneous and mucosal pallor, a dramatic reduction in rat mobility, without clinical sign of paralysis. Rats were not moribund, having sleek hair, not dehydrated but with hyporeactivity. Once the biological diagnosis of profound anemia and the clinical observations made rats were sacrificed the same day for ethical reasons. The inventors have also established that said functional inactivation may also result in a moderate anemia, wherein the percentage of hematocrit is comprised between 30 and 20%, or in a slight anemia, wherein the percentage of hematocrit is comprised between 35 and 30%.

By the term “neutralizing antibodies” as used herein is meant antibodies or fragment thereof that are able to target the endogenous erythropoietin protein (EPO) of the subject and to hamper its biological activity.

As used herein, the term “subject” refers to a vertebrate, preferably a mammal. If it is a mammal, the subject will be preferably a human for the gene therapy applications or bone marrow transplantation of the method of the invention, but may also be a domestic livestock, pet animal, or a laboratory animal, and the subject will be preferably a non-human for the screening applications.

More preferably, the term “subject” refers to a rodent, such as rat or mouse.

As used herein “endogenous erythropoietin” refers to the erythropoietin encoded by the genome of the subject and expressed in said subject. As an example, such endogenous erythropoietin refers to SEQ ID NO:1, when the subject is a rat (Rattus norevegicus).

As used herein “erythropoietin protein” refers to an erythropoietin protein selected among vertebrate species, preferably among mammal species, such as dog rabbit, mouse, rat (SEQ ID NO:1), pig, primate or human.

As used herein “erythropoietin protein derivative” refers to a polypeptide having a percentage of identity of at least 10%, 15%, 20%, 30%, 40%, 55%, 60%, 65%, 70%, 75%, 80%, 85%, 87.5%, 90%, 92.5%, 95%, 96%, 97%, 98%, 99%, 99.5%, 99.9% with an erythropoietin protein expressed in a vertebrate species or fragment thereof, preferably of at least 70%, as an example of at least 85%, and more preferably of at least 95% with an erythropoietin protein expressed in a vertebrate species or a fragment thereof, preferably in a mammal species.

As used herein “fragments” refers to polypeptides having a length of at least 6 amino acids, preferably at least 10 amino acids and more preferably of at least 15 amino acids.

Preferably, said erythropoietin protein derivative presents an increased immunogenicity profile compared to the erythropoietin protein of reference according to the antigenic prediction described in WELLINGS et al. (FEBS, vol. 188(2), p: 215-218, 1985). Preferably, said increased immunogenicity profile is located in at least in one antibody-accessible region of the EPO derivative. One of skill in the art can easily determined antibody-accessible region, as an example from the hydrophobicity profile (e.g. obtained according to Kyte & Doolittle method, see for example http://www.expasy.org/tools/protscale.html) of said derivative, wherein the antibody-accessible regions correspond hydrophilic regions. This increase of immunogenicity is obtained by mutation(s) of nucleic acid sequence encoding erythropoietin protein, which mutation(s) are selected in a group consisting of naturally occurring mutation, genetically engineered mutation, chemically induced mutation, physically induced mutation. Preferably, mutation is induced by recombinant DNA techniques known in the art. For example, it may include among others, site directed mutagenesis or random mutagenesis of DNA sequence which encodes said protein. Such methods may, among others, include polymerase chain reaction (PCR) with oligonucleotide primers bearing one or more mutations (Ho et al., 1989) or total synthesis of mutated genes (Hostomsky et al., 1989). These methods can be used to create variants which include, e.g., deletions, insertions or substitutions of residues of the known amino acids sequence of the heterologous protein of the invention. PCR mutagenesis using reduced Taq polymerase fidelity can also be used to introduce random mutations into a cloned fragment of DNA (LEUNG et al., Technique, vol. 1, p: 11-15, 1989). Random mutagenesis can also be performed according to the method of MAYERS et al., Science, vol. 229, p: 242, 1985). This technique includes generations of mutations, e.g., by chemical treatment or irradiation of single-strand DNA in vitro, and synthesis of a complementary DNA strand.

As an example, said erythropoietin protein derivative for inactivation of endogeneous EPO in rat has an amino acids sequence selected in the group comprising SEQ ID NO:2, SEQ ID NO:3, SEQ ID NO: 4, SEQ ID NO: 5, and SEQ ID NO: 6, and more preferably said erythropoietin protein derivative is SEQ ID NO:2, SEQ ID NO: 5 or SEQ ID NO: 6.

Preferably, said erythropoietin protein derivative is SEQ ID NO:2 or SEQ ID NO: 6.

As another example, said erythropoietin protein derivative for inactivation of endogeneous EPO in mouse has an amino acids sequence selected in the group comprising SEQ ID NO:2, SEQ ID NO:3, SEQ ID NO: 4 and SEQ ID NO: 6, and more preferably said erythropoietin protein derivative is SEQ ID NO:2 or SEQ ID NO: 6.

Advantageously, said erythropoietin protein or derivative has a percentage of identity of at least 10%, 15%, 20%, 30%, 40%, 55%, 60%, 65%, 70%, 75%, 80%, 85%, 87.5%, 90%, 92.5%, 95%, 96%, 97%, 98%, 99%, 99.5%, 99.9% with the endogenous erythropoietin, preferably of at least 60%, as an example of at least 70%, and most preferably of at least 80% with the endogenous erythropoietin.

As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p: 482, 1981), by using the local homology algorithm developed by NEDDLEMAN and WUNSCH (J. Mol. Biol., vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p: 1792, 2004). To get the best local alignment, one can preferably used BLAST software, with the BLOSUM 62 matrix, or the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

As used herein amino acids sequences having a percentage of identity of at least 50%, preferably at least 60%, as an example at least 70%, and most preferably at least 80% after optimal alignment, means amino acids sequences having with regard to the reference sequence, modifications such as deletions, truncations, insertions, chimeric fusions, and/or substitutions, specially point mutations, the amino acids sequence of which presenting at least 50%, 60%, 70% or 80% of identity after optimal alignment with the amino acids sequence of reference (e.g., the amino acids sequence of the endogenous erythropoietin), respectively.

The nucleic acid sequence encoding for said erythropoietin protein or derivative can be used to transiently transfect or transform subject cells, or can be integrated into the subject cell chromosome. Preferably, however, the nucleic acid sequence can include sequences that allow its replication and stable or semi-stable maintenance in the subject cell. Such sequences for use in various eukaryotic cells are well known in the art. Generally, it is preferred that replication sequences known to function in subject cells of interest be used.

Preferably the nucleic acid sequence encoding for erythropoietin protein or derivative contains all the genetic information needed to direct the expression of said erythropoietin protein or derivative in at least one cell of the subject, preferably in at least one APC cell of the subject such as promoter sequences, regulatory upstream elements, transcriptional and/or translational initiation, termination and/or regulation elements. Various promoters, including ubiquitous or tissue-specific promoters, and inducible and constitutive promoters may be used to drive the expression of erythropoietin protein or derivative. Preferred promoters for use in mammalian host cells include strong viral promoters from polymoma virus, Simian Virus 40 (SV40), adenovirus, retroviruses, hepatitis B virus, herpes simplex virus (HSV), Rous sarcoma virus (RSV), mouse mammary tumor virus (MMTV), and most preferably cytomegalovirus (CMV), cytomegalovirus enhancer plus chicken beta-actin promoter (CAG) but also heterologous mammalian promoters such as the β-actin promoter, phosphoglycerate kinase (PGK) promoter, epithelial growth factor 1 α(EGF1α) promoter, albumin promoter, creatine kinase promoter, methall-thionein promoter. In preferred embodiments, the promoters are chosen among cytomegalovirus early promoter (CMV IEP), Rous sarcoma virus long terminal repeat promoter (RSV LTR), myeloproliferative sarcoma virus long terminal repeat (MPSV LTR), simian virus 40 early promoter (SV40 IEP), and major late promoter of the adeovirus. Alternatively, other eukaryotic promoters are suitable for such use, including elongation factor one-alpha (EF1-α) promoter, creatinine kinase promoter, albumine promoter, phosphoglycerate kinase promoter. Inducible promoters such as tertacycline promoters could also be used. Transcription of the gene encoding the heterologous protein can be increased by inserting an enhancer sequence into the vector. Many enhancer sequences are now known from mammalian genes (globin, elastase, albumin, and insulin) or from eukaryotic cell virus (SV40, CMV). The disclosed vectors preferably also contain a polyadenylation signal. All of the above mentioned regulation sequences are operably linked to provide optimal expression of the transgene.

As used herein, the term “agent” refers to a compound selected in the group comprising viruses, liposomes, antibodies, parasites, bacteriae, funguses, and fragments thereof, and naked nucleic acid sequence encoding said erythropoietin protein or derivative.

In a preferred embodiment, the method of the invention comprises the step (a) of administrating to said subject at least one agent selected in the group comprising viruses, parasites, bacteriae and fungunses, which genome comprises at least one nucleic acid sequence encoding for an erythropoietin protein or derivative and regulation sequences necessary to direct the expression of said erythropoietin protein or derivative.

By “the affective amount of agent” it means the number of moieties that is administrated to the subject. For a virus, this amount includes the recombinant particles that encode and express erythropoietin (EPO) or derivative and incomplete, empty or EPO-non-coding virus particles that “contaminate” the viral stock.

The effective amount of said at least one agent and at least one erythropoietin protein or derivative or nucleic acid sequence encoding said erythropoietin or derivative for triggering the production of neutralizing antibodies against the endogenous erythropoietin (EPO) in said subject can be determined as described in PCT application WO 03/013594 (page 23, lines 9 to 24; page 26, line 3 to 13; page 29, line 12 to page 30, line 4; which are herein incorporated by reference).

Advantageously, said agent is a virus, which is preferably selected among adenovirus, adenovirus associated virus, retrovirus, lentivirus, pox virus, vaccinia virus, or fragments thereof. Preferred virus is adenovirus, which is selected among wild type adenovirus and recombinant adenovirus, and more preferably recombinant adenovirus.

Recombinant adenovirus have advantages for use as transgene expression systems, including tropism for both dividing and non-dividing cells, minimal pathogenic potential, ability to replicate to high titer for preparation of vector stocks, and the potential to carry large inserts (see e.g., BERKNER, Curr. Top. Micro. Immunol., vol. 158, p: 39-66, 1992; JOLLY, Cancer Gene Therapy, vol. 1, p: 51-64, 1994). Adenovirus vectors can accommodate a variety of transgenes of different sizes. For example, about an eight (8) kb insert can be accommodated by deleting regions of the adenovirus genome dispensable for growth (e.g., E3). Development of cell lines that supply non-dispensable adenovirus gene products in trans (e.g., E1, E2a, E4) allows insertion of a variety of transgenes throughout the adenovirus genome (see e.g. GRAHAM et al., J. Gen. Virol., vol. 36, p: 59-74, 1977; IMLER et al., Gene Therapy, vol. 3, p: 75-84, 1996). Preferably the components of the adenovirus transgene expression system (i.e. the transcription unit, E3 cassette, E4 cassette) are configured on a single adenovirus vector. Preferably, the adenovirus vector is replication-defective. This is not intended to be limiting of the transgene expression systems, since the components can be configured in a number of ways to meet the intended use. For example, in one preferred embodiment, the adenovirus vector comprises a transcription unit comprising the transgene (i.e the nucleic acid sequence encoding said heterologous protein) inserted into the E1a, E1b region of adenovirus. In this embodiment, the adenovirus vector further comprises the E3 cassette and the E4 cassette configured in positions corresponding to the E3 and E4 regions of adenovirus, respectively. The adenovirus vector largely comprises adenovirus genome sequences, and further comprising at least a portion of an adenovirus E3 region and an E4 ORF3 and at least one portion of E4. Preferably, the adenovirus vector is incapable of productively replicating in host cells unless co-infected with an adenovirus helper virus or introduced into a suitable cell line supplying one or more adenovirus gene products in trans (e.g., 293 cells). Adenoviruses with larger deletion of the viral genome (MAIONE et al., Proc. Natl. Acd. Sci. USA, vol. 98, p: 5986-91, 2001) can also be used for the applications described in the invention. An adenovirus vector according to the invention can belong to any one of the known six human subgroups, e.g., A, B, C, D, E, or F, wherein a preferred series of serotypes (all from subgroup D) includes Ad9, Ad15, Ad17, Ad19, Ad20, Ad22, Ad26, Ad27, Ad28, Ad30, or Ad39. Preferred serotypes include the Ad2 and Ad5 serotypes. Additionally, chimeric adenovirus vectors comprising combinations of Ad DNA from different serotypes are within the scope of the present invention. Adenoviruses from other species (porcine, ovine, bovine, canine, murine etc. . . . ) can also be used for the same purpose. The adenovirus vectors of the invention can be made in accordance with standard recombinant DNA techniques.

In general, the vectors are made by making a plasmid comprising a desired transcription unit inserted into a suitable adenovirus genome fragment. The plasmid is then co-transfected with a linearised viral genome derived from an adenovirus vector of interest and introduced into a recipient cell under conditions favoring homologous recombination between the genomic fragment and the adenovirus vector. Preferably, the transcription unit is engineered into the site of an adenovirus E1 deletion. Accordingly, the transcription unit is inserted into the adenoviral genome at a pre-determined site, creating a recombinant adenoviral vector. The recombinant adenovirus vector is further recombined with additional vectors comprising desired E3 and/or E4 cassettes to produce the adenovirus vectors. The recombinant adenovirus vectors are encapsidated into adenovirus particles as evidenced by the formation of plaques in standard viral plaque assays. Preparation of replication-defective adenovirus stocks can be accomplished using cell lines that complement viral genes deleted from the vector, (e.g., 293 or A549 cells containing the deleted adenovirus E1 genomic sequences). After amplification of plaques in suitable complementing cell lines, the viruses can be recovered by freeze-thawing and subsequently purified using cesium chloride centrifugation. Alternatively, virus purification can be performed using chromatographic techniques. Examples of such techniques can be found for example in published PCT application WO/9630534, and ARMENTANO et al. (Human Gene Therapy, vol. 6, p: 1343, 1993; each reference incorporated herein by reference).

As an example, the effective amount of recombinant adenovirus comprising at least one nucleic acid sequence encoding for an erythropoietin protein or derivative that has to be administrated to a rat or mouse for triggering the production of neutralizing antibodies against the endogenous erythropoietin (EPO) is equal or below 2.10¹⁰ particles, preferably equal or below 10¹⁰ particles, as an example equal or below 5.10⁹ particles, and more preferably equal or below 10⁹ particles.

As an example, the effective amount of recombinant adenovirus comprising at least one nucleic acid sequence encoding for an erythropoietin protein or derivative that has to be administrated to a rat or mouse for triggering the production of neutralizing antibodies against the endogenous erythropoietin (EPO) is greater than 10⁵ particles, preferably greater than 10⁶ particles, as an example greater than 10⁷ particles, and more preferably greater than 5.10⁷ particles.

The determination of the particles concentration in a viral stock can be performed according to well known methods such as the measurement of optical density by absorbance at 260 or 280 nm or the counting of particles by electron microscopy.

Preferably, a particle corresponds to an infectious particle.

The administration of said agent and said erythropoietin protein or derivative, or nucleic acid sequence encoding said erythropoietin protein or derivative is performed via a technique chosen among intravenous injection, intravaginal injection, intrarectal injection, intramuscular injection, subcutaneous, intradermic injection, gene gun delivery, oral or nasal delivery. Preferably, the administration is performed via subcutenous, intradermic, or intramuscular injection, and more preferably via subcutaneous injection. Single injection or multiple injections at the same or at different loci can be performed.

The step a) of administration of said agent and said erythropoietin protein or derivative, or nucleic acid sequence encoding said erythropoietin protein or derivative can be repeated once or several time.

In fact, the inventors have established that a second immunization enable to reinforce the anemia phenotype.

According to another embodiment, said at least one agent corresponds to an adjuvant that enhance the production of neutralizing antibodies. Adjuvants are well known from one of skill in the art and include, as examples, (1) aluminium salts (alum), such as aluminium hydroxide, aluminium phosphate, aluminium sulfate, etc.; (2) oil-in-water emulsion formulations, such as for example MF59 (WO 90/14 837), SAF, Ribi™ adjuvant system (Ribi Immunochem, Hamilton, Mont. USA); (3) saponin adjuvants; (4) complete Freunds adjuvant (CFA) and incomplete Freunds adjuvant (IFA); and (5) cytokines such as interleukines (Il-1, Il-2, etc.), macrophage colony stimulating factor (M-CSF), tumor necrosis factor (TNF).

Advantageously, the method of the invention thus comprises the step (a) of co-administrating to said subject at least one adjuvant and at least one erythropoietin protein or derivative, or at least one nucleic acid sequence encoding said erythropoietin protein or derivative, preferably the co-administration to said subject of at least one adjuvant and at least one erythropoietin protein derivative.

In another embodiment, the method of the invention is a method of screening and identifying compounds acting as EPO agonists, and further comprising the step (b) of administrating at least one compound to the subject.

As used herein, the term “EPO agonist” refers to a compound, which can occupy at least one cellular receptor of erythropoietin, and having the same effect than erythropoietin on at least one action selected in the group comprising production of red blood cells, mitogenesis, modulation of calcium influx into smooth muscle cells and neural cells, production of erythrocytes, and hyperactivation of thrombocytes.

Advantageously, said compound is selected in the group comprising peptides, polypeptides, small-molecules, nucleic acids, lipids, and carbohydrates.

Preferably, the method of the invention further comprises the step (c) of comparing the subject's phenotype before and after the step (b).

As used herein the term “phenotype” refers to the physical appearance and the observable properties of the subject that are produced by the interaction of the genotype with the environment.

Advantageously, the subject's phenotype corresponds to the production of red blood cells, the mitogenesis, the modulation of calcium influx into smooth muscle cells and neural cells, the production of erythrocytes, and/or the hyperactivation of thrombocytes.

Such subject's phenotype can be identified by method well known from one of skill in the art such as numeration of red blood cell in subject blood samples.

More preferably, the method of the invention further comprises the step (d) of selecting the compounds that reactivate at least one function of erythropoietin that has been inactivated by the EPO neutralizing antibodies produced by the subject after the administration step (a).

Said at least one function is selected in the group comprising production of red blood cells, mitogenesis, modulation of calcium influx into smooth muscle cells and neural cells, production of erythrocytes, and hyperactivation of thrombocytes. Preferably, said at least one function corresponds to the production of erythrocytes.

The identification of the reactivation of at least one function of erythropoietin in the subject can be done by method well known from one of skill in the art such as numeration of red blood cell in subject blood samples for the production of red blood cells.

More advantageously, the method of the invention comprises the step (b') of administrating at least one known EPO mimetic as a control. EPO mimetics are known from the skilled person and includes, as examples, the polypeptides described in U.S. Pat. No. 4,703,008; the agonists described in U.S. Pat. No. 5,767,078; the peptides which bind to the erythropoietin receptor as described in U.S. Pat. Nos. 5,773,569 and 5,830,851; and small-molecule mimetics as described in U.S. Pat. No. 5,835,382.

In still another embodiment, the method of the invention is a method for screening and identifying compounds acting as oxygen transporters, and further comprising the step (b) of administrating at least one compound to said subject.

Preferably, the method of the invention further comprises the step (c) of comparing the subject's phenotype before and after the step (b).

Preferably, the subject's phenotype refers to the physical appearance and the observable properties of the subject which can results from anemia such as a low blood oxygen concentration, tachycardia, debility, digestive disorder or vertigo.

Such subject's phenotype can be identified by method well known from one of skill in the art.

More preferably, the method of the invention further comprises the step (d) of selecting the compounds that reverts the phenotype of anemia resulting from the production of EPO neutralizing antibodies by the subject after the administration step (a).

More advantageously, the method of the invention comprises the step (b′) of administrating at least one known oxygen transporter as a control. Oxygen transporters are known from the skilled person and includes, as examples, the oxygen transporter described in patent applications EP 1,466,649, FR 2,799,466, and in US 2003/130,487.

In still another embodiment, the method of the invention is a method for treating and/or preventing pathology associated with abnormal red blood cells in a subject and further comprises the step of (b) administrating bone marrow cells to said subject enabling the obtaining of normal red blood cells.

Pathology associated with abnormal red blood cells are well known from one of skill in the art. As an example, one can cites sickle-cell anemia, thalassemia or G6PD deficiency.

The bone marrow cells can be from said subject or from another subject. If said bone marrow cells are from the same subject, said cells have been genetically modified in order to correct the disorder.

Advantageously, the method of the invention thus comprises the step (c) of administrating an EPO mimetics to said subject simultaneously or following the step (b).

The method of the invention enables to reduce considerably the quantity of abnormal red blood cells in the blood of said subject. The method can be realized one or more times to the subject.

The subject, preferably a non-human, obtained by the method of the invention of producing functional inactivation of endogenous erythropoietin is also in the scope of the invention. Such subject mammal is preferably chosen among domestic life stock, pet animals as previously described or among laboratory animals like for example, mouse, rat, rabbit, Chinese pig, hamster, dwarf pig, monkeys and others. More preferably, the animal is a rat.

The use of the subject obtained by the above described method to isolate spleen cells from said subject that expresses antibody directed against said endogenous erythropoietin to make hybridoma(s) is also in the scope of the invention. Alternatively, the biological fluid of the subject of the invention can be used to prepare serum and/or polyclonal antibodies.

The obtained erythropoietin neutralizing antibodies or derivatives, preferably the obtained monoclonal erythropoietin neutralizing antibodies or derivatives are also in the scope of the invention. As used herein, an “antibody derivative” refer to humanized antibodies, single-chain Fvs (scFv), single-chain antibodies, single domain antibodies, Fab fragments, F(ab′)2 fragments, or disulfide-linked Fvs (sdFv).

“Humanized” forms of nonhuman (e.g., rat) antibodies are chimeric antibodies that contain minimal sequence derived from nonhuman immunoglobulin. For the most part, humanized antibodies are human immunoglobulins (recipient antibody) in which residues from a hypervariable region of the recipient are replaced by residues from a hypervariable region of a nonhuman species (donor antibody) such as mouse, rat, rabbit or nonhuman primate having the desired specificity, affinity, and capacity. In some instances, framework region (FR) residues of the human immunoglobulin are replaced by corresponding nonhuman residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, JONES et al. (Nature, vol. 321, p: 522-525, 1986); RIECHMANN et al. (Nature, vol. 332, p: 323-329, 1988); and PRESTA (Curr. Op. Struct. Biol., vol. 2, p: 593-596, 1992).

Finally, the use of erythropoietin neutralizing antibodies or derivatives in a method for functional inactivation of endogenous erythropoietin comprising the step (a) of administrating an effective amount of at least one erythropoietin neutralizing antibody or derivative to a subject is also in the scope of the invention. Preferably said method is a method for treating and/or preventing pathology associated with abnormal red blood cells in a subject and further comprises the step (b) of administrating bone marrow cells to said subject enabling the obtaining of normal red blood cells.

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See AUSUBEL et al., Current Protocols in Molecular Biology, Eds., John Wiley & Sons, Inc. New York, 1995; Remington's Pharmaceutical Sciences, 17^{th} ed., Mack Publishing Co., Easton, Pa., 1985; and SAMBROOK et al., Molecular cloning: A laboratory manual 2^{nd} edition Cold Spring Harbor Laboratory Press-Cold Spring Harbor, N.Y., USA, 1989).

Unless defined otherwise, all technical and scientific terms used herein have the same meaning as is commonly understood by one of the skill in the art to which this invention belongs.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLES

### I EPO Mutants Constructions

1) Design of an EPO Mutant

The mature erythropoietin consists of 166 amino acid residues, along with 3 oligosaccharide chains. The protein itself has a mass of 18 KDa while the human glycosylated protein has a mass of 30 KDa. This mature erythropoietin is obtained from an EPO precursor being composed of one main domain EPO (26-192) which is a four helix cytokine domain that is involved in the erythrocyte differentiation and the maintenance of a physiological level of circulating erythrocyte mass, and the peptide-signal domain (1-26).

We performed an analysis of immunogenicity of Rat EPO precursor by using antigenic prediction described by WELLING et al. (FEBS, vol. 188 (2), page 215-218, 1985). The prediction based on individual score for each amino acid along the peptide sequence allows the determination of antigenic region profile in the protein.

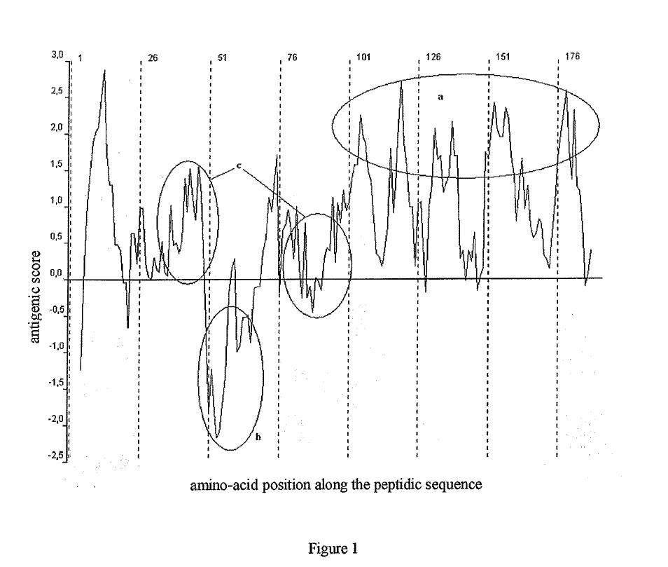

The immunogenicity analysis has revealed an average immunogenic protein with several picks of immunogenicity (see FIG. 1). More specifically, the immunogenicity analysis has identified a large area between 110 and 185 which is highly antigenic (see FIG. 1, b), a region between 45 and 65 which shows very low antigenic values (see FIG. 1, a) and two regions which are in between these values (see FIG. 1, c).

The main goal of our strategy was to introduce variability in the sequence of the EPO precursor in order to create new antigenic regions able to generate a cross reactive antibody response targeting the endogenous EPO. Thus, different erythropoietin derivatives have been generated by introducing modifications, which were spread all over the sequence to avoid non specific immune response and absence of cross-reactive immune response targeting the endogenous protein.

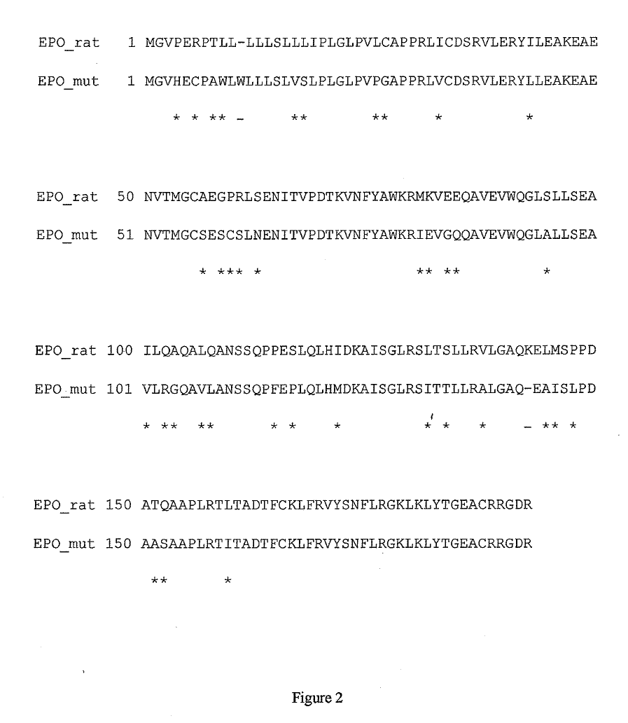

The comparison of amino acid sequences of rat and one mutant EPO precursors shows that the two proteins share 81% of their sequence (see FIG. 2). Moreover, the nucleotidic alignment of mutant and rat cDNA shows an identity of 79.27% between both sequences.

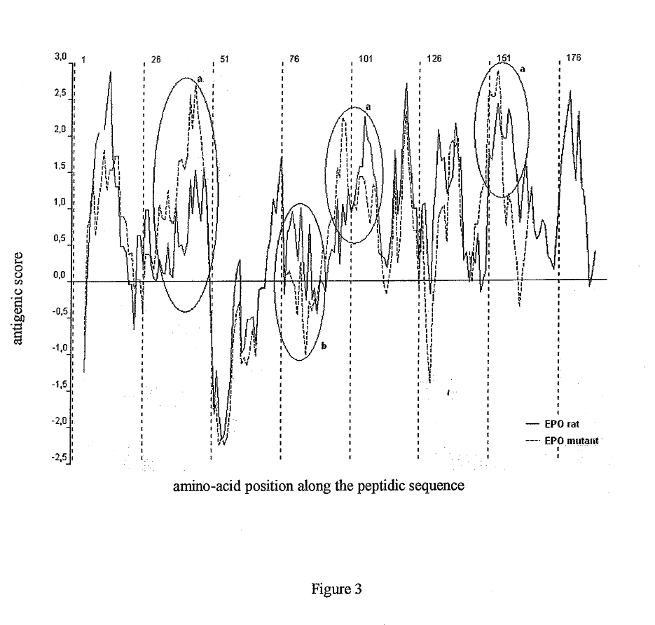

The comparison of the immunogenicity profiles revealed several differences of the mutant protein with the rat EPO precursor (see FIG. 3). More specifically, the introduced variability has induced a strong immunogenicity increase in few regions (see FIG. 3, a).

The comparison of amino acid sequences of rat and the second (EPOmut1, SEQ ID NO:6) and third (EPOmut2, SEQ ID NO:5) mutants shows that these mutants share 80.83 and 94.79% of their sequence.

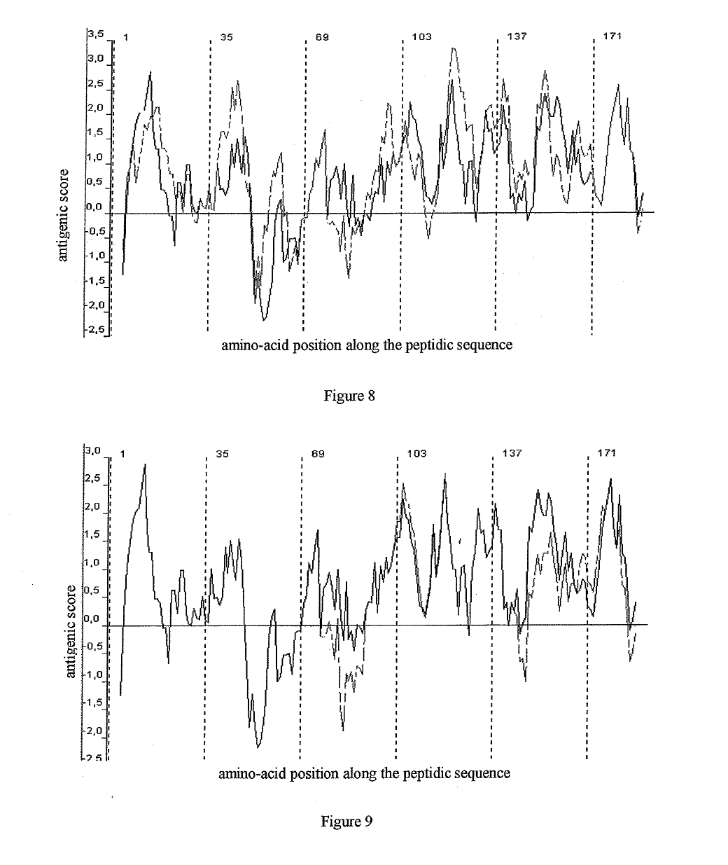

The comparison of the immunogenicity profiles revealed several differences of the EPO Mut1 with the rat EPO precursor (see FIG. 8), while said profiles comparison revealed only slight differences between the EPO Mut2 and the rat EPO precursor (see FIG. 9).

In conclusion, the introduced variability has induced a strong immunogenicity increase in few regions for EPO mut and EPO Mut1 variants, whereas it has induced only a slight immunogenicity increase in few regions for EPO Mut2 variant.

2) Construction of Recombinant E1-E3 Deleted Adenovirus Vectors:

Fragments of nucleotides coding for the rat EPO, EPOmut, EPOmut1 or EPO mut2 were cloned in an adenoviral shuttle vector CAG pShuttle (ADDGENE) in which the homologous EPO sequence is under the control of the CAG promoter (i.e., a combination of chicken beta-actin promoter and cytomegalovirus immediate-early enhancer) and the SV40 polyadenylation signal for obtaining the CAG pShuttle.EPOrat, CAG pShuttle.EPOhom, CAG pShuttle.EPOhom1 or CAG pShuttle.EPOhom2 plasmids.

The CAG pShuttle.EPOrat CAG pShuttle.EPOhom, CAG pShuttle.EPOhom1 or CAG pShuttle.EPOhom2 plasmids were then digested by Pme I (BIOLABS) and an homologous recombination with the pAdEasy-1 adenoviral backbone plasmid (STRATAGENE) was carried out in Escherichia Coli Cells for the generation of the recombinant adenovirus Ad-EPOrat, Ad-EPOMut, Ad-EPOMut1 or Ad_EPOMut2. An Ad.RSV.null, generated by recombination between pAdEasy-1 and the pShuttle.RSV, was used as a control.

Propagation of the recombinant adenoviral vectors was then performed in HEK293 cells. Viral stocks were prepared by infection of the 293 cell line, purified and concentrated by a double cesium chloride gradient, by an exclusion chromatography step to remove the cesium salts, aliquoted, and stored in 10% glycerol at −80° C. Titers of the viral stocks were determined by limiting dilution on plaque assays using 293 cells and expressed as PFU. The total number of viral particles was quantified by optical density at 260 nm of an aliquot of the virus stock diluted in virion lysis solution (0.1% SDS, 10 mM Tris-HCl, 1 mM EDTA).

The absence of E1A sequence and of host cell DNA contaminant was confirmed for each adenoviral vector using PCR.

### II Functional Inactivation of the Endogenous EPO Protein in Rat

1) Induction of a Functional Inactivation of the Endogenous EPO Protein in Rat after Vaccination with the Mutant EPO

Females OFA rats (Sprague Dawley), 8 weeks old were obtained from Charles River, France, and housed at the CERFE animal facility (Genopole, Evry, France). All experiments were conducted in concordance with the local ethical and scientific policies.

All animals were bred in negative pressure isolators and placed in confined room until downgrading was validated (absence of infectious particles in animal blood).

Animals were acclimatized for 10 days before the beginning of procedures.

For this study rats were individually labelled.

All manipulation in presence of adenovirus particles were made in a confined room in compliance with the French GMO regulations.

The 10 weeks animals were subdivided in 3 groups: PBS, AdNull (recombinant adenovirus E1 and E3 deleted but free of the transgene sequence), Ad-EPO-rat (E1 and E3 deleted recombinant adenovirus coding for the EPO rat cDNA), Ad-EPOMut, Ad-EPOMut 1 and Ad-EPOMut2 treated groups (E1 and E3 deleted recombinant adenovirus coding for EPO homolougous cDNAs).

Isoflurane-anesthetized animals were subcutaneously injected with 1.10⁹ infectious particles of Ad-EPOMut, Ad-EPOMut1, Ad-EPOMut2, Ad-EPOrat, AdNull in 100 μl of PBS (GIBCO, INVITROGEN) or PBS alone using a 18 G needle gauge.

They were observed every week, and followed by a blood cells numeration once or twice a month. The blood samples were obtained from by puncture of the retro-orbital sinus using a Pasteur pipette. 1 ml of blood was collected per rat once a month.

For the peripheral blood hematologic measurements, 300 μl of the 1 ml collected blood was dropped immediately in a 1.5 ml polypropylene tube containing 3 μl of EDTA 0.5M (GIBCO) for haematological measurement. The measurement was made between 30 minutes and 4 hours after the puncture using the MS9 animal blood counter, (MELET SCHLOESING).

The rest of blood was centrifuged in a 1.5 ml polypropylene tube at 21° C. between 30 min and 1 h after the puncture, 12 minutes at 2000 rpm. Sera were collected carefully with a p200 tip, stocked in a 1.5 ml propylene tube and immediately stored at −20° C.

The number of White Blood Cell (WBC), and Red Blood Cell (RBC), the value of hematocrit (HCT) and the number of Platelet (PLT) have been determined for each blood sample. The results of the PBS, AdNull and Ad-EPOMut treated groups are shown in table 1.

The FIGS. 10, 11, 12 and 13 show the evolution in the value of hematocrit (HCT %) depending on the time after injection for Ad-EPOmut, Ad-EPOmut1, Ad-EPOmut2 and Ad-EPOrat treated groups respectively.

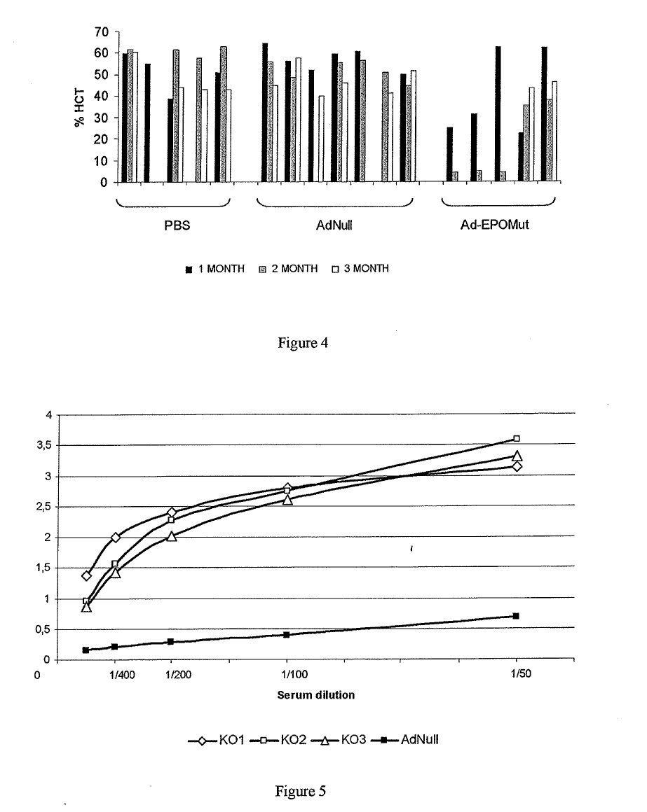

The results show that the rats injected with the Ad-EPOMut displayed transient elevations in hematocrit (HCT) during the first month (see FIG. 4 and Rats n° 15 and 17 in Table 1 with respectively 62.6% and 62.2% of hematocrit). This high elevation of hematocrit was followed by a profound anemia the next month. This anemia persisted during the time of experience for 3 rats (see Table 1, wherein rats n° 13, 14, 15 showed a percentage of hematocrit of 4.1%, 5% and 4.5% at the second month after injection) whereas 2 rats started an anemia that was be reversed the third month (rat n° 16 had an 35.2% of hematocrit the second month and 43.6% the third month, n° 17 showed 37.7% the second month and 46.3% the third month). The phenotype observed for the three rats with a hematocrit less than 5% was a pale blood.

Among the rats treated with Ad-EPOmut, 4 rats on 8, representing 50% of them, showed a EPO-KO phenotype 2 months after the prime vaccination (FIG. 10). 3 of these rats had a hematocrit level under 15%. The others rats showed a transient phenotype, with a decrease in HCT levels at 1.5 month and values subsequently increased the second month.

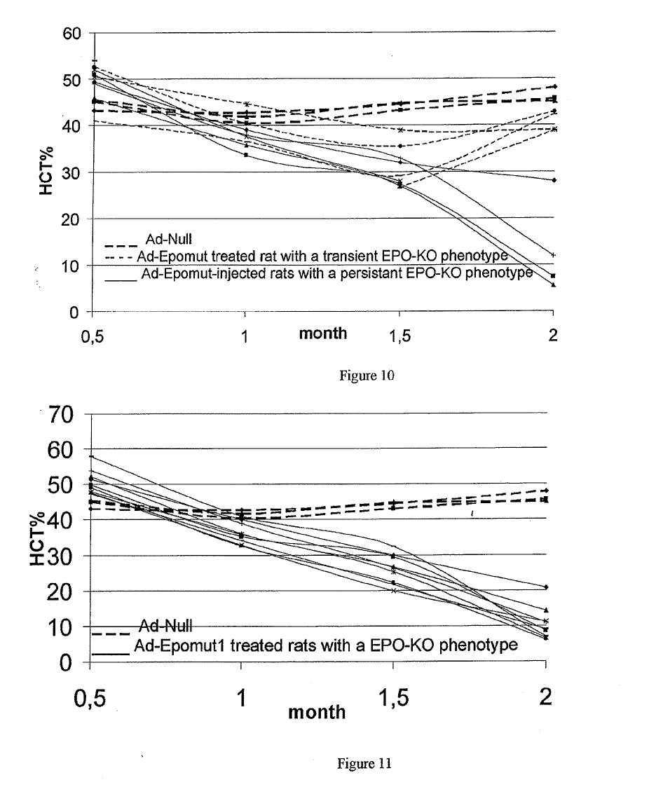

Among the rats treated with Ad-EPOmut1, all of them show a very rapid drop in hematocrit level with a continued decrease to profound anemic values (10.5±4.6%; see FIG. 11).

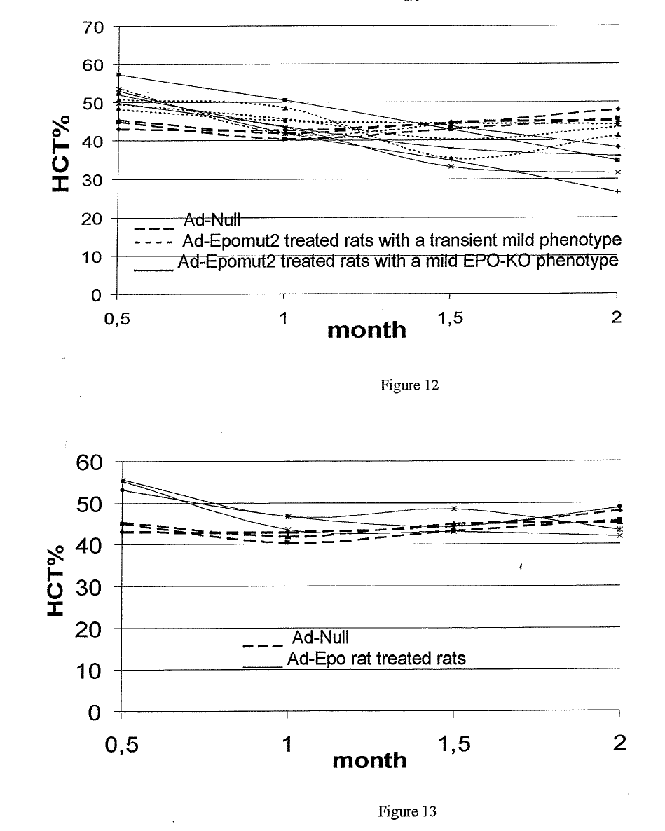

Among the rats treated with Ad-EPOmut2, all of them show a very rapid drop in hematocrit level with a continued decrease to anemic values (10.5±4.6, see FIG. 12) in 3 over the six injected rats with hematocrit values of (39±6.13% the second month) correction.

For negative control, no significant variation of red cells concentration or hematocrit level was observed in rats PBS, AdNull (49.15%+/−8.61; 60.8%+/−2.35; 47.35%+/−8.59 for PBS group and 56.95%+/−5.53; 51.76%+/−4.64; 46.75%+/−6.73 for AdNull group during the three months) or Ad-EPOrat (see FIG. 13) groups.

Finally, the hematocrit evolution represented in FIGS. 4, 10, 11 and 12 suggests that the injection with Ad-EPOMut, Ad-EPOMut1, Ad-EPOMut2 has an influence on rat EPO tolerance by the organism. The observed anemia might result from humoral responses directed against rat EPO (endogenous), which leads to a decrease in blood red cells and thus in hematocrit. Moreover, it can be observed that said decrease depend upon the difference in immunogenicity between the rat EPO and the EPO mutants, with the greatest increased in immunogenicity (EPOmut and EPOmut1) associated with the greatest decrease in hematocrit value.

TABLE 1
blood cell numeration of rat treated with Ad-EPOMut, AdNull or PBS during 3 months.

| | | 1 MONTH | | | | 2 MONTHS | | | | 3 MONTHS | | | |
|---|---|---|---|---|---|---|---|---|---|---|---|---|---|
| | | WBC | RBC | HCT | PLT | WBC | RBC | HCT | PLT | WBC | RBC | HCT | PLT |
| | N^{o} | (10³/μl) | (10⁶/μl) | (%) | (10³/μl) | (10³/μl) | (10⁶/μl) | (%) | (10³/μl) | (10³/μl) | (10⁶/μl) | (%) | (10³/μl) |
| PBS | 1 | 7.1 | 10.75 | 59.6 | 274 | 7 | 11.04 | 61.6 | 361 | 8.83 | 10.84 | 60.2 | 321 |
| | 2 | 6.29 | 6.87 | 38.7 | 557 | 5.39 | 10.94 | 61.4 | 315 | 6.13 | 7.95 | 43.9 | 296 |
| | 3 | COAG | | | | 9.16 | 10.27 | 57.4 | 526 | 7.01 | 7.48 | 42.6 | 556 |
| | 4 | 3.24 | 8.91 | 50.6 | 448 | 5.79 | 11.28 | 62.8 | 298 | 6.5 | 7.8 | 42.7 | 314 |
| | 5 | 3.59 | 8.4 | 47.7 | 167 | COAG | | | | COAG | | | |
| | mean | | | 49.15 | | | | 60.8 | | | | 47.35 | |
| | SD | | | +/−8.61 | | | | +/−2.35 | | | | +/−8.59 | |
| AdNull | 6 | 6.68 | 11.67 | 64.5 | 418 | 9 | 10.2 | 55.5 | 624 | 6.32 | 8.1 | 44.5 | 468 |
| | 7 | 7.43 | 9.86 | 55.9 | 538 | 4.54 | 8.63 | 48.3 | 237 | 8.88 | 10.44 | 57.6 | 456 |
| | 8 | 8.4 | 9.07 | 51.8 | 482 | | | | | SACRIF | | | |
| | 9 | 7.8 | 10.81 | 59.3 | 762 | 5.51 | 9.96 | 55.1 | 494 | 7.46 | 8.11 | 45.8 | 689 |
| | 10 | 5.6 | 10.73 | 60.4 | 451 | 7.43 | 9.89 | 56.2 | | SACRIF | | | |
| | 11 | COAG | | | | 6.63 | 9.25 | 50.8 | 602 | 5.54 | 7.59 | 41.3 | 716 |
| | 12 | 7.46 | 8.79 | 49.8 | 1042 | 8.37 | 7.98 | 44.7 | 748 | 7.92 | 9.27 | 51.6 | 753 |
| | mean | | | 56.95 | | | | 51.76 | | | | 46.75 | |
| | SD | | | +/−5.53 | | | | +/−4.64 | | | | +/−6.73 | |
| EPOMut | 13 | 2.43 | 3.71 | 25 | 821 | 1.96 | 0.76 | 4.1 | 128 | SACRIF | | | |
| | 14 | 7.1 | 5.19 | 31.3 | 523 | 5.03 | 0.93 | 5 | 411 | SACRIF | | | |
| | 15 | 4.5 | 10.02 | 62.6 | 431 | 0.73 | 0.7 | 4.5 | 54 | SACRIF | | | |
| | 16 | 6.96 | 7.39 | 41.8 | 316 | 6.81 | 5.88 | 35.2 | 359 | 8.39 | 8.03 | 43.6 | 652 |
| | 17 | 11.59 | 10.15 | 62.2 | 1116 | 7.91 | 6.28 | 37.7 | 391 | 7.93 | 8.28 | 46.3 | 486 |
| | mean | | | 44.58 | | | | 17.3 | | | | 44.95 | |
| | SD | | | +/−20.07 | | | | +/−17.51 | | | | +/−1.91 | |

A second immunisation was done for EPO-Null, EPOMut and EPOMut 1 treated groups with the same protocol than the one used for the first immunisation, and nearly three months after said first immunisation.

The value of hematocrit was determined as previously.

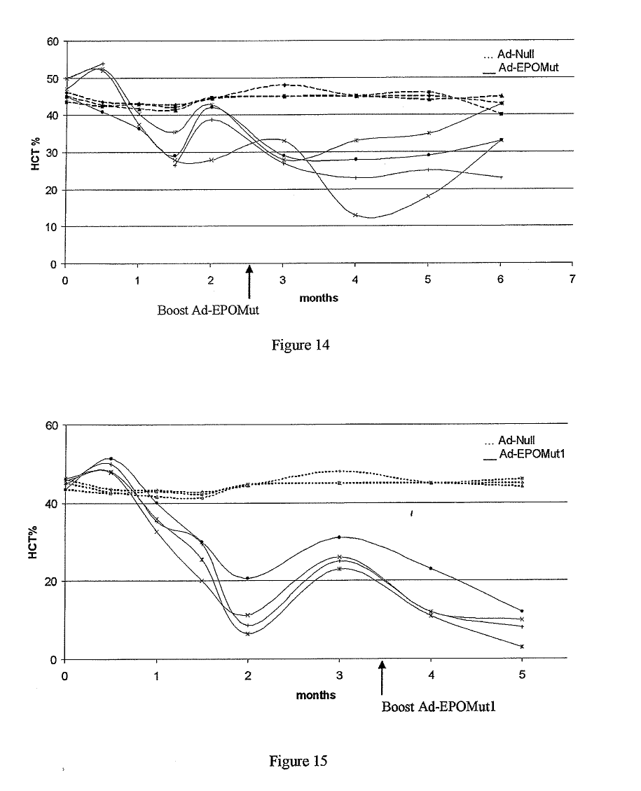

The FIGS. 14 and 15 show the evolution in the value of hematocrit (HCT %) depending on the time after the first injection (time 0) and after a second immunization (arrow) for Ad-EPOmut and Ad-EPOmut1 treated groups respectively.

The results show that, excepted rats sacrificed after the second month for ethical reasons, the rats presented a transient response after the third month: Ad-EPOmut treated group: 29.25±1.31HCT (see FIG. 14) and Ad-EPO-mut1 treated group: 26.25±3.40% HCT (see FIG. 15).

In Ad-epoMut1 group, we observed an immediate drop for all rats (FIG. 15, 8.25±3.86%) the fifth month. This mutant allowed the obtention of 100% of EPO-KO rat

In Ad-EPOmut injected rats, we obtained 75% of EPO-KO phenotype I month after boost with a transient phenotype. 2 month after the boost, 1 rat over the 4 injected-rats among those who had a transient response following the prime immunisation, showed a subsequent decrease in hematocrit values (see FIG. 14).

2) Presence of a Humoral Response Against Endogenous EPO

A ninety six well plate (NUNC MAXISORB) was coated with 50 μl of recombinant rat EPO (SIGMA-ALDRICH) at 10 μg/ml diluted in coating buffer (0.1M carbonate/bicarbonate buffer, pH 9.6) overnight at 4° C.

The plate was washed 3 times with 300 μl per well of PBS containing 0.05% TWEEN® 20 (SIGMA-ALDRICH), and then saturated with PBS containing 3% BSA (SIGMA-ALDRICH) for 90 minutes at room temperature.

The contents of wells were flicked out and, after 3 washes, 100 μl of a serial dilution of serum from KO EPO rat (Ad-EPOMut treated rats) and negative control rats (AdNull treated rats) was added and incubated for 1 hour at room temperature and 1 hour at 37° C. Serum were diluted in PBS/1.5% BSA from 1/50 to 1/800.

The wells were washed 4 times and incubated with 100 μl of horseradish peroxidase-conjugated goat anti-rat IgG and IgM (JACKSON IMMUNORESEARCH LABORATORIES) diluted at 1/10000 in PBS/BSA 1.5% for 1 hour at room temperature. After 5 washes 100 μl of TMB (BD BIOSCIENCE) was added in each well for 20 minutes in the dark and the reaction was stopped using 50 μl of HCl 1M. The absorbance was measured (μQUANT spectrophotometer, BIO-TEK INSTRUMENTS) 30 minutes after stopping the reaction, at 450 nm against a reference blank at 570 nm.

The results show that the sera from AdNull did not contain antibodies reactive against endogenous rat EPO by contrast with the sera from Ad-EPOMut which contained antibodies reactive against endogenous rat EPO (see FIGS. 5 and 6).

3) Dosage Erythropoietin in Rat Serum

EPO (erythropoietin) quantification levels in KO-EPO rat sera was performed by an ELISA using “Quantikine mouse/rat EPO immunoassay” (R&D system, ref. MEP00) according to the manufacturer's instructions. A polystyrene microtiter plates coated with monoclonal antibodies specific for mouse/rat EPO were incubated with standards, positive control, and samples for 2 hours at room temperature on a horizontal orbital microplate shaker set at 200 rpm. After washing 5 times, an HRP-linked monoclonal antibody specific for mouse/rat EPO is added to the wells for 2 hours. Following washes, a substrate solution is added to the wells during 30 minutes and the enzyme reaction was stopped with Stop Solution. Optical density was assessed at 450 nm and to correct wavelength a measure was performed at 570 nm. The serum EPO levels were then determined using a standard curve by comparing their OD values.

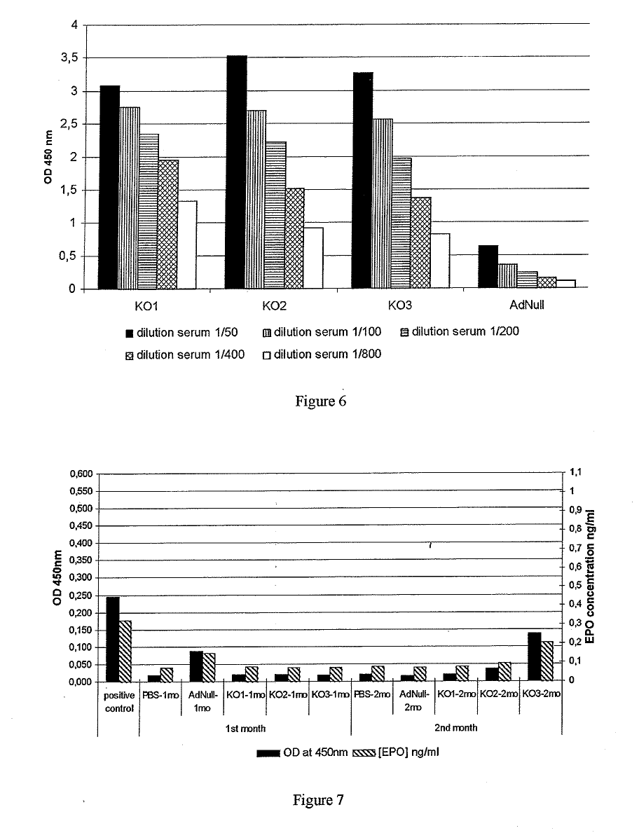

The results are shown in table 2 and in FIG. 7.

TABLE 2
EPO cytokine Detection in KO-EPO rat sera. Values obtains from
ELISA at 450 nm and determination of the EPO concentration by
the standard curve (ng/ml)

| | | OD at 450 nm | [EPO] ng/ml |
|---|---|---|---|
| | positive control | 0.244 | 0.177 |
| 1st month | PBS | 0.017 | 0.040 |
| | AdNull | 0.086 | 0.081 |
| | KO1 | 0.021 | 0.042 |
| | KO2 | 0.019 | 0.041 |
| | KO3 | 0.018 | 0.040 |
| 2nd month | PBS | 0.021 | 0.042 |
| | AdNull | 0.016 | 0.039 |
| | KO1 | 0.020 | 0.042 |
| | KO2 | 0.038 | 0.052 |
| | KO3 | 0.137 | 0.112 |

Normally, the serum EPO level increase exponentially as the hematocrit decreases and as hypoxia is very important. The results show that no EPO detectable level was observed in PBS, or AdNull rat, but also for KO-EPO rats as shown in table 2 and FIG. 7 with most of the values under the minimum detection dose of 0.05 ng/ml despite the hypoxia induced by the complete neutralization of the species differentiation and absence of blood cells.

On the contrary, the two other rats in Ad-EPOMut group (n° 16 and 17, table 1), which show transient and mild neutralized phenotype have presented an OD at 450 nm of 0.801 and 1.139 means 0.515 ng/ml and 0.719 ng/ml of EPO concentration, respectively.

Because of the presence of anti-ratEPO antibodies in serum, the relationship between hematocrit, hypoxia and EPO level is likely to be complex.

Finally, the results have established that the anti-rat EPO antibodies in serum were functionally active, leading to block the endogenous EPO and its functionality. With low free EPO levels in the serum, the proliferation of erythropoietic precursor cells was inhibited, as measured by CFU-E clonogenic progenitors (CFU-E means colony forming unit-species), red blood cells were not produce any more and thus animals presented a severe anaemia. Even if this anaemia induces a production of EPO in KO-EPO rats, the neutralizing antibodies block this EPO immediately before it interacts with EPO receptor.

### III Functional Inactivation of the Endogenous EPO Protein in Mice

1) Induction of a Functional Inactivation of the Endogenous EPO Protein in Mice after Vaccination with the Mutant EPO.

In order to generalize, the induction of functional inactivation of endogenous EPO protein after vaccination with mutants EPO to other species, experiments were conducted on different mice strains.

66 males C57B16/J mice, 8 weeks old were obtained from Charles River, France, and housed at the CERFE animal facility (Genopole, Evry, France). All experiments were conducted in concordance with the local ethical and scientific policies.

All animals were bred in negative pressure isolators and placed in confined room until downgrading was validated (absence of infectious particles in animal blood).

Animals were acclimatized for 10 days before the beginning of procedures.

For this study mice were individually labelled.

All manipulation in presence of adenovirus particles were made in a confined room in compliance with the French GMO regulations.

Isoflurane-anesthetized animals were subcutaneously (SC) or intramuscularly (IM) injected with 0.5.10⁹, 1.10⁹, 2.10⁹ or 5.10⁹ infectious particles of Ad-EPOMut or AdNull in 100 μl of PBS (GIBCO, INVITROGEN) using a 18 G needle gauge.

They were observed every week, and followed by a blood cells numeration twice a month. The blood samples were obtained by puncture of the retro-orbital sinus using a Pasteur pipette. 0.2 ml of blood was collected per mouse twice a month.

For the peripheral blood hematologic measurements, 300 μl of the 1 ml collected blood was dropped immediately in a 1.5 ml polypropylene tube containing 3 μl of EDTA 0.5M (Gibco) for haematological measurement. The measurement was made between 30 minutes and 4 hours after the puncture using the MS9 animal blood counter, (MELET SCHLOESING).

The rest of blood was centrifuged in a 1.5 ml polypropylene tube at 21° C. between 30 min and 1 h after the puncture, 12 minutes at 2000 rpm. Sera were collected carefully with a p200 tip, stocked in a 1.5 ml propylene tube and immediately stored at −20° C.

The value of hematocrit (HCT %) has been determined for each blood sample. The results are shown in table 3.

TABLE 3
Induction of a functional inactivation of the endogenous EPO protein in mice after
vaccination with the Ad-EPOMut.

| | 1 month | 1.5 month | 2 month | 2.5 month | 3 month | 3.5 month | 4 month |
|---|---|---|---|---|---|---|---|
| Ad-Null SC 5 × 10e8 ip | 45.1 | 46 | 48.6 | 48 | 47.8 | 56 | — |
| Ad-Null SC 5 × 10e8 ip | 42.9 | 51 | 47.3 | 46 | 48.6 | 53 | 50 |
| Ad-Null SC 5 × 10e8 ip | 45.2 | 50 | 48 | 45 | 44.4 | | — |
| Ad-Null SC 1 × 10e9 ip | 43.1 | 42 | 40.3 | 44 | 45 | | 40.6 |
| Ad-Null SC 1 × 10e9 ip | 45 | 45 | 44.7 | 45 | 45.6 | 45 | 44.2 |
| Ad-Null SC 1 × 10e9 ip | 46.1 | — | — | 46 | 45.5 | | 42.1 |
| Ad-Null SC 2 × 10e9 ip | 43.6 | 45 | 47.9 | 46 | 45.3 | 49 | 41 |
| Ad-Null SC 2 × 10e9 ip | 43.9 | 50 | 46.7 | 46 | 46.2 | | 46.7 |
| Ad-Null SC 2 × 10e9 ip | 41.3 | 49 | — | 45 | — | 44 | 43.8 |
| Ad-Null IM 5 × 10e8 ip | 42.9 | 50 | 48 | 47 | 46.8 | 55 | |
| Ad-Null IM 5 × 10e8 ip | 42.6 | 50 | 46.6 | sacrified | | | |
| Ad-Null IM 5 × 10e8 ip | 41.5 | 55 | 52 | 50 | 49.2 | 49 | 41.8 |
| Ad-Null IM 1 × 10e9 ip | 43.9 | — | 45.9 | 48 | 49.9 | 50 | |
| Ad-Null IM 1 × 10e9 ip | 43 | 50 | 43.7 | 45 | — | — | 43.4 |
| Ad-Null IM 1 × 10e9 ip | 47 | 49 | 48.5 | 50 | 47.4 | 53 | |
| Ad-Null IM 2 × 10e9 ip | 45.9 | 50 | 49.3 | 50 | 47.6 | 48 | 41.6 |
| Ad-Null IM 2 × 10e9 ip | 42.5 | | 47.1 | 55 | 49.3 | | 40.4 |
| Ad-Null IM 2 × 10e9 ip | 43.2 | 52 | 46.2 | 45 | 45.8 | | 42.3 |
| Ad-EPOmut SC 5 × 10e8 ip | 90 | — | | | | | |
| Ad-EPOmut SC 5 × 10e8 ip | 91 | | | | | | |
| Ad-EPOmut SC 5 × 10e8 ip | 57.4 | 39 | 27.9 | 35 | 35.1 | 36 | 36.6 |
| Ad-EPOmut SC 5 × 10e8 ip | 90 | — | | | | | |
| Ad-EPOmut SC 1 × 10e9 ip | 49.8 | 35 | 32.2 | 35 | 39.5 | — | 37.4 |
| Ad-EPOmut SC 1 × 10e9 ip | | | | | | | |
| Ad-EPOmut SC 1 × 10e9 ip | 48.1 | 32 | 23.1 | 29 | 32.5 | 41 | 32.8 |
| Ad-EPOmut SC 1 × 10e9 ip | 45.7 | 28 | 16.6 | 22 | 24.9 | 33 | 26.8 |
| Ad-EPOmut SC 2 × 10e9 ip | 90 | | | | | | |
| Ad-EPOmut SC 2 × 10e9 ip | 56 | 39 | 35.9 | — | 44.2 | 50 | 44.8 |
| Ad-EPOmut SC 2 × 10e9 ip | 50.5 | 40 | 37.2 | 45 | 42.8 | 48 | 42.5 |
| Ad-EPOmut SC 2 × 10e9 ip | 92 | 50 | | | | | |
| Ad-EPOmut IM 5 × 10e8 ip | 50.8 | 35 | 25.9 | | | | |
| Ad-EPOmut IM 5 × 10e8 ip | 61.3 | 57 | 46.1 | 47 | 47.4 | 55 | 43.6 |
| Ad-EPOmut IM 5 × 10e8 ip | 57.6 | 52 | 25.7 | | | | |
| Ad-EPOmut IM 5 × 10e8 ip | 64.1 | 66 | 61.2 | 64 | 61.9 | 70 | 62.2 |
| Ad-EPOmut IM 1 × 10e9 ip | 50.4 | 46 | 31.5 | 38 | 42.2 | 55 | 53 |
| Ad-EPOmut IM 1 × 10e9 ip | 40.9 | 41 | 40 | 41 | 42.3 | — | 40.5 |
| Ad-EPOmut IM 1 × 10e9 ip | 54.5 | 39 | 28.6 | 25 | 36.1 | 50 | 35.4 |
| Ad-EPOmut IM 1 × 10e9 ip | 54.2 | 47 | 35 | | | | |
| Ad-EPOmut IM 2 × 10e9 ip | 67.2 | 80 | 90 | 70 | 67 | 67 | |
| Ad-EPOmut IM 2 × 10e9 ip | 57.5 | 42 | 18 | 24 | 30.7 | 45 | 34.5 |
| Ad-EPOmut IM 2 × 10e9 ip | | | | | | | |
| Ad-EPOmut IM 2 × 10e9 ip | 53.4 | 44 | 28.2 | 28 | 33.8 | 44 | 38.3 |

The results show that the mice injected with the Ad-EPOMut displayed transient elevations in hematocrit (HCT) during the first month. This high elevation of hematocrit was followed by a profound anemia the next month, said anemia being also more pronounced for the subcutaneous Ad-EPOmut injected mice. After the second month, all the values are stable or increased for the subcutaneous and intramuscular Ad-EPOmut injected mice. Negative control values remained in normal HCT range (41%-52%) for both type of injection. The results also established that the injection of 1.10⁹ infectious particles of Ad-EPOMut enable to obtain the greatest decrease of HCT for both injection modes.

Finally, the results confirmed those observed for the rats and established that the subcutaneous adenovirus injection induced a more pronounced phenotype than the one observed with intramuscular injection. This difference may result from differential expression of the EPOmut encoded by the adenovirus between both types of injection.

2) Impact of a Vaccinal Boost on the Mice Phenotype

The same protocol was tested on C57BL6/J and CD1 mice.

C57BL6/J mice were injected subcutaneously with Ad-EPOmut or Ad-EPOmut1 with a dose of 1.10⁸, 2.10⁸ and 4-5.10⁸ ip/mouse.

CD1 mice were injected subcutaneously with Ad-EPOmut1 at a dose of 1.10⁸, 2.10⁸ and 4-5.10⁸ ip/mouse.

Control groups were injected subcutaneously with AdNull or Ad-EPO murine at the same doses.

The results have shown that 4 over the 6 vaccinated CD1 mice injected by the subcutaneous route with Ad-EPOmut1 at 1.10⁸ ip/mouse had an EPO-KO phenotype 1.5 month post injection with hematocrit levels below 20%.

Similarly, decreased hematocrit levels, even if less pronounced, were observed in 2 over the 5 injected C57BL6/J mice.

A second immunisation for C57BL6/J and CD1 mice and for EPO-Null, EPOMut and EPOMut1 treated groups was done three months after the first injection and with the same protocol.

The results have shown that the second round of immunization induce again a decrease of the hematocrit level in CD1 mice, and induce an EPO-KO phenotype 1 month post injection with hematocrit levels below 20% for 2 over 5 C57BL6/J mice.

Consequently, the second round of vaccination enable to increase the prevalence of the EPO-KO phenotype in all AD-EPOMut1 treated mice.

3) Vaccination with a Mix of EPO Mutants can Increase the Functional Inactivation of EPO Protein.

A mix (Ad-EPOmix) of Ad-EPOmut1 and Ad-EPOmut at a ratio of 1:1, was injected subcutaneously on C57B16/J mice (5.10⁸ ip/mouse) as described previously. As a control, mice were injected with the same dose of Ad-Null.

A second injection was done on said mice 1.2 month after the first immunization using the same conditions.

Hematocrit levels (HCT) were determined by blood cells numeration at 1 month, 2 month, 2.1 month, 2.5 month and 3 months. The results are presented in table 4.

TABLE 4
HCT levels of C57B16/J mice followed during 3 months
after a prime immunization with a mix of
Ad-EPOmut1 and Ad-EPOmut (Ad-EPOmix)

| | | | | 2.5 | 3 |
|---|---|---|---|---|---|
| | 1 month | 2 months | 2.1 months | months | months |
| Ad-Null | 54.4 | 50.7 | 46.2 | sacrificed | |
| | 50.9 | 53.4 | 55 | 54 | 52.4 |
| | 48.6 | 50 | 53.2 | 53.2 | 53.2 |
| Ado-EPOMix | 65.6 | 60.4 | 66.3 | 75 | 70.4 |
| 5 × 10⁸ ip | 47.9 | 11.2 | sacrificed | | |
| | 51.5 | 15.8 | 8.8 | sacrificed | |
| | 65 | 50 | 42.7 | 32.4 | 28.7 |
| | 46 | 18.3 | 15.7 | 25.2 | 21 |

The results show that 2 mice over the 5 injected with EPO mix showed a rapid drop in HCT percentage with HCT values at 11% and 15.8%. Finally, 4 mice over 5 had an EPO-KO phenotype on the third months (see Table 4).

Mix of Ad-EPOmut1 and Ad-EPOmut (Ad-EPOmix) at a ratio of 1:1, was also tested on C57B16/J TPO−/− mice at 3 to 5 months age old, using the same protocol as described before. The prime injection and the boost were both at 2.10⁸ and 5.10⁸ ip/mouse. The blood cells numeration was done at 1 month, 2 month, 2.1 month, 2.5 month and 3 month. As a control, Ad null injection was used.

The results are shown in Table 5

TABLE 5
Results of HCT evolution in C57B16/J/J TPO−/− mice at different
doses followed during 3 months after a prime immunization with
a mix of Ad-EPOmut1 and Ad-EPOmut (Ad-EPOmix).

| | 1 month | 2 month | 2.1 month | 2.5 month | 3 month |
|---|---|---|---|---|---|
| Ad-null 5 × | 47.2 | 48.2 | sacrified | | |
| 10e8 ip | 46.4 | 50 | 51.5 | sacrified | |
| | 43.1 | 47.7 | 49.2 | 49.9 | 52.8 |
| | 45.5 | 29.2 | 16.7 | 52.3 | 46.9 |
| | 46.4 | 48.8 | 50.7 | 50.4 | 53.2 |
| Ad-EPOmix | 43.6 | 8.2 | sacrified | | |
| 2 × 10e8 ip | 43.7 | 8.6 | sacrified | | |
| | 51.6 | 4.2 | sacrified | | |
| | dead | | | | |
| | dead | | | | |
| | 59.8 | dead | | | |
| | 45.7 | 44.6 | 33.5 | 31.1 | 34 |
| | 83.8 | 95 | 93.4 | 90.9 | 89.6 |
| | 53.7 | 16.4 | 6.2 | sacrified | |
| | 68.9 | 54.6 | 28.1 | 9.9 | 9.1 |
| | 62.5 | — | 30.4 | 14.6 | 25 |
| | 52.8 | 10.4 | 5.3 | sacrified | |
| | 78 | 80.4 | 77.1 | 85.7 | dead |
| | 51.7 | 3.6 | sacrified | | |
| | 13.4 | sacrified | | | |
| Ad-EPOmix | 28.3 | 15.6 | 18.5 | 12.9 | sacrified |
| 5 × 10e8 ip | 42.1 | 6.5 | sacrified | | |
| | 42.6 | 39.8 | 17.7 | dead | |
| | 74.9 | 67.8 | 71 | 62 | 83.6 |
| | 36.4 | 4.8 | sacrified | | |
| | 42.9 | dead | | | |
| | 42.3 | 16.1 | 17.5 | | |
| | 39.4 | 4.2 | sacrified | | |
| | — | 11.7 | sacrified | | |

The results show that C57B16/J TPO−/− mice, injected with a mix Ad-EPomut+AdEPomut 1 treatment at 2×10⁸ ip/mouse and boosted 1.1 month later with the same dose as the prime immunization, induced a neutralized-EPO phenotype in 50% of mice with an EPO-KO phenotype three months after the first vaccination. 8 out of the 15 injected mice showed an EPO-KO phenotype in the Ad-mix injected group at 2×10e8 ip/mouse. 6 mice had less than 10% HCT, 2 mice with had HCT values between 15-25%.

C57B16/J TPO−/− mice, injected with a mix Ad-EPomut+AdEPomut1 treatment at 5×10⁸ ip/mouse and boosted 1.1 month later with the same dose as the prime immunization, induced a neutralized-EPO phenotype in 7 mice out of the 9 injected animals at three month. 3 mice had a less than 10% HCT, 4 mice had an HCT between 10-25% (see Table 5).

In conclusion, the results on C57B16/J strain, both on wild type animals or TPO−/−, were greatly improved when AdEPOmix was used as a prime-boost regimen.

4) Correlation Between the Hematocrit Values and the Concentration of Epo Antibodies Present in TPO−/− C57B16/J Mice.

The serum from Ad-EPOmix treated. TPO−/− C57B16/J mice were tested by direct ELISA to detect anti-mouse Epo antibodies as described previously for anti-rat Epo antibodies.

The results indicate that, as observed with rats, the hematocrit values are correlated with the concentration of Epo antibodies present in TPO−/− C57B16/J mice.

We claim:

1. A method for functional inactivation of endogenous erythropoietin (EPO) in a subject comprising the step of (a) co-administrating to said subject at least one agent and at least one erythropoietin protein or derivative, or at least one nucleic acid sequence encoding said erythropoietin protein or derivative, said agent being administrated simultaneously, sequentially or separately with said at least one erythropoietin protein or derivative, or at least one nucleic acid sequence encoding said erythropoietin protein or derivative, wherein said at least one agent and at least one erythropoietin protein or derivative, or at least one nucleic acid sequence encoding said erythropoietin protein or derivative are administrated in an effective amount for triggering the production of neutralizing antibodies against the endogenous erythropoietin (EPO) in said subject.

2. The method of claim 1, wherein said method results in the disturbance of erythropoietin functions such as the production of red blood cells, mitogenesis, modulation of calcium influx into smooth muscle and neural cells, production of erythrocytes, hyperactivation of platelets, and/or production of thrombocytes.

3. The method of claim 1, wherein said method results in an anemia.

4. The method of claim 2, wherein said method results in a profound anemia with a percentage of hematocrit below 20%.

5. The method of claim 3, wherein said method results in a moderate anemia with a percentage of hematocrit comprised between 30% and 20%.

6. The method of claim 3, wherein said method results in a slight anemia with a percentage of hematocrit comprised between 30% and 35%.

7. The method of claim 1, wherein said subject is a vertebrate.

8. The method of claim 7, wherein said subject is a mammal.

9. The method of claim 1, wherein said erythropoietin protein is an erythropoietin protein selected among vertebrate species.

10. The method of claim 9, wherein said erythropoietin protein is an erythropoietin protein selected among mammal species, such as erythropoietin from dog, rabbit, mouse, rat, pig, primate or human.

11. The method of claim 1, wherein said erythropoietin protein derivative is a polypeptide having a percentage of identity of at least 10% with an erythropoietin protein expressed in a vertebrate species or fragment thereof.

12. The method of claim 1, wherein said erythropoietin protein derivative is a polypeptide having a percentage of identity of at least 70% with an erythropoietin protein expressed in a vertebrate species or fragment thereof.

13. The method of claim 1, wherein said erythropoietin protein derivative is a polypeptide having a percentage of identity of at least 85% with an erythropoietin protein expressed in a vertebrate species or fragment thereof.

14. The method of claim 1, wherein said erythropoietin protein derivative is a polypeptide having a percentage of identity of at least 95% with an erythropoietin protein expressed in a vertebrate species or fragment thereof.

15. The method of claim 1, wherein said erythropoietin protein derivative presents an increased immunogenicity profile compared to the erythropoietin protein of reference, preferably in at least in one antibody-accessible region.

16. The method of claim 1, wherein said erythropoietin protein derivative for inactivation of endogeneous EPO in rat has an amino acids sequence selected in the group comprising SEQ ID NO:2, SEQ ID NO:3, SEQ ID NO: 4, SEQ ID NO: 5, and SEQ ID NO: 6, and preferably said erythropoietin protein derivative is SEQ ID NO:2, SEQ ID NO: 5 or SEQ ID NO: 6.

17. The method of claim 1, wherein said erythropoietin protein derivative for inactivation of endogeneous EPO in mouse has an amino acids sequence selected in the group comprising SEQ ID NO:2, SEQ ID NO:3, SEQ ID NO: 4 and SEQ ID NO: 6, and preferably said erythropoietin protein derivative is SEQ ID NO:2 or SEQ ID NO: 6.

18. The method of claim 1, wherein said erythropoietin protein derivative has the amino acids sequence of SEQ ID NO:2.

19. The method of claim 1, wherein said erythropoietin protein or derivative has a percentage of identity of at least 10% with the endogenous erythropoietin.

20. The method of claim 1, wherein said erythropoietin protein or derivative has a percentage of identity of at least 50% with the endogenous erythropoietin.

21. The method of claim 1, wherein said erythropoietin protein or derivative has a percentage of identity of at least 80% with the endogenous erythropoietin.

22. The method of claim 1, wherein said agent is selected in the group comprising viruses, liposomes, antibodies, parasites, bacteriae, funguses.

23. The method of claim 22, wherein said method comprises the step of (a) administrating to said subject at least one agent selected in the group comprising viruses, parasites, bacteriae and fungunses, which genome comprises at least one nucleic acid sequence encoding for an erythropoietin protein or derivative and regulation sequences necessary to direct the expression of said erythropoietin protein or derivative.

24. The method of claim 23, wherein said agent is a virus selected in the group comprising adenovirus, adenovirus associated virus, retrovirus, lentivirus, pox virus, vaccinia virus, or fragments thereof.

25. The method of claim 24, wherein said virus is an adenovirus.

26. The method of claim 25, wherein said adenovirus is a recombinant adenovirus.

27. The method of claim 26, wherein the effective amount of recombinant adenovirus comprising at least one nucleic acid sequence encoding for said erythropoietin protein or derivative is equal or below 2.10¹⁰ particles.

28. The method of claim 26, wherein the effective amount of recombinant adenovirus comprising at least one nucleic acid sequence encoding for said erythropoietin protein or derivative is equal or below 10¹⁰ particles.

29. The method of claim 26, wherein the effective amount of recombinant adenovirus comprising at least one nucleic acid sequence encoding for said erythropoietin protein or derivative is greater than 10⁶ particles.

30. The method of claim 1, wherein said administration step is performed via a technique chosen among intravenous injection, intravaginal injection, intrarectal injection, intramuscular injection, intradermic injection, subcutaneous injection.

31. The method of claim 1, wherein said administration step (a) is repeated once or several times.

32. The method of claim 1, wherein said method is a method of screening and identifying compounds acting as EPO agonists, and further comprising the step (b) of administrating at least one compound to the subject.

33. The method of claim 32, wherein said compound is selected in the group comprising peptides, polypeptides, small-molecules, nucleic acids, lipids, and carbohydrates.

34. The method of claim 32, wherein said methods further comprises the step (c) of comparing the subject's phenotype before and after the step (b).

35. The method of claim 34, wherein said subject's phenotype corresponds to the production of red blood cells, the mitogenesis, the modulation of calcium influx into smooth muscle cells and neural cells, the production of erythrocytes, and/or the hyperactivation of thrombocytes.

36. The method of claim 35, wherein said methods further comprises the step (d) of selecting the compounds that reactivate at least one function of erythropoietin that has been inactivated by the EPO neutralizing antibodies produced in the subject after the administration step (a).

37. The method of claim 34, wherein said method further comprises the step (b') of administrating at least one known EPO mimetic as a control.

38. The method of claim 1, wherein said method is a method of screening and identifying compounds acting as oxygen transporters, and further comprising the step (b) of administrating at least one compound to said subject.

39. The method of claim 38, wherein said compound is selected in the group comprising peptides, polypeptides, small-molecules, nucleic acids, lipids, and carbohydrates.

40. The method of claim 39, wherein said method further comprises the step (c) of comparing the subject's phenotype before and after the step (b).

41. The method of claim 40, wherein said subject's phenotype corresponds to the physical appearance and the observable properties which can results from anemia and selected in the group comprising low blood oxygen concentration, tachycardia, debility, digestive disorder and vertigo.

42. The method of claim 41, wherein said method further comprises the step (d) of selecting the compounds that reverts the phenotype of anemia resulting from the production of neutralizing antibodies by the subject after the administration step (a).

43. The method of claim 38, wherein said method further comprises the step (b') of administrating at least one known oxygen transporter as a control.

44. The method of claim 1, wherein said method is a method for treating and/or preventing pathology associated with abnormal red blood cells in a subject and further comprises the step (b) of administrating allogeneic bone marrow cells bone marrow cells or autologous bone marrow cells after a corrective gene therapy process or other correcting agents to said subject enabling the obtaining of normal red blood cells.

45. The method of claim 44, wherein the pathology associated with abnormal red blood cells is selected in the group comprising sickle-cell anemia, thalassemia and G6PD deficiency (other red cell pathological conditions).

46. The method of claim 45, wherein said method further comprises the step (c) of administrating an EPO mimetic to said subject simultaneously or following the step (b).

47. A non human vertebrate which can be obtained by the method as defined in claim 1.

48. A neutralizing antibody directed against erythropoietin isolated from a subject as defined in claim 47.


SEQUENCE LISTING (6 sequences)

SEQ ID NO: 1 - protein, 185 aa, Rattus norvegicus
MGVPERPTLLLLLSLLLIPLGLPVLCAPPRLICDSRVLERYILEAKEAEILQAQALQANSSQPPESLQLHIDKAISGLRSLTSLLRVLGAQKELMSPPDATQAAPLRTLTADTFCKLFRVYSNFLRGKLKLYTGEACRRGDRAASAAPLRTITADTFCKLFRVYSNFLRGKLKLYTGEACRRGDR

SEQ ID NO: 2 - protein, 192 aa, Artificial sequence
  Erythropoietin derivative
MGVHECPAWLWLLLSLVSLPLGLPVPGAPPRLVCDSRVLERYLLEAKEAENVTMGCSESCSLNENITVPDTKVNFYAWKRIEVGQQAVEVWQGLALLSEAVLRGQAVLANSSQPFEPLQLHMDKAISGLRSITTLLRALGAQEAISLPDAASAAPLRTITADTFCKLFRVYSNFLRGKLKLYTGEACRRGDR

SEQ ID NO: 3 - protein, 206 aa, Artificial sequence
  Erythropoietin derivative
MCEPAPPKPTQSAWHSFPECPALLLLLSLLLLPLGLPVLGAPPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 4 - protein, 195 aa, Artificial sequence
  Erythropoietin derivative
MGARGRLALLPLALLCLLVLALGLPVLGAPARLICDSRVLERYILEAKEAENVTMGCAEGCSLGENITVPDTKVNFHHWKKSEAGRHAVEVWQGLALLSEAMLRSQALLANSSQLPETLQVHVDKAVSGLRSLTSLLRALGVQKEAVSPPEAASSAAPLRTVAADTLCKLFRIYSNFLRGKLKLYTGEACRRGDR

SEQ ID NO: 5 - protein, 192 aa, Artificial sequence
  Erythropoietin derivative
MGVPERPTLLLLLSLLLIPLGLPVLCAPPRLICDSRVLERYILEAKEAENVTMGCAEGPRLSENITVPDTKVNFYAWKRMEVEEQAIEVWQGLSLLSEAILQAQALLANSSQPPETLQLHIDKAISGLRSLTSLLRVLGAQKELMSPPDTTPPAPLRTLTVDTFCKLFRVYANFLRGKLKLYTGEVCRRGDR

SEQ ID NO: 6 - protein, 193 aa, Artificial sequence [sequence omitted]